(19) United States
(12) Patent Application Publication
(10) Pub. No.: US 2012/0046623 A1
(43) Pub. Date: Feb. 23, 2012

(54) Body Fluid Collection Pouch with Anti-Return Valve

(75) Inventor: Jérôme Bordeau, Le Chene (FR)

(73) Assignee: B. BRAUN MEDICAL SAS, Boulogne-Billancourt (FR)

(21) Appl. No.: 13/255,061

(22) PCT Filed: Mar. 1, 2010
(86) PCT No.: PCT/EP10/01247

§ 371 Date: Nov. 8, 2011

(30) Foreign Application Priority Data
Mar. 6, 2009 (FR) 0951409

(51) Int. Cl. A61M 1/00

(52) U.S. Cl. 604/317

(57) ABSTRACT

The invention relates to a collection pouch (1) for bodily fluids, comprising in particular welds (7, 8) forming an anti-reflux valve. Said collection pouch (1) comprises in addition an entrance (6) to a collection compartment (3) and an admission orifice (4) that are offset. The device has a direct application in medical care.

The present invention relates to a fluid collection pouch. More specifically, the present invention allows collecting body fluids such as, for example, blood or urine. In addition to this particularity, the invention also makes it possible to store solutions used for infusion, for washing or rinsing wounds or medical instruments.

The use of body fluid collection pouches, as described e.g. in patent application EP 1 825 838, is well known. This document describes a collection pouch onto which an anti-return valve is fixed whose principal function is to prevent any reflux whatsoever of the liquid contained within said collection pouch. By way of illustration, we may cite the draining and collection of urine during a urinary catheterization procedure, also known as bladder catheterization. During the implementation of such a bladder catheterization procedure, the device often comprises not only a urinary catheter, but also a collection pouch with an anti-reflux valve. Such a valve prevents the return of fluid, i.e. of the urine collected and stored in the collection pouch, into the urinary catheter and eventually the bladder.

However, due to the geometric complexity and the number of components of the devices according to the state of the art, manufacturing such devices requires advanced and specific know-how as well as expensive workforce and manufacturing equipment. More specifically, the collection pouches according to the state of the art comprise in particular a pouch and an anti-reflux valve, the latter being constituted by generally more than one piece which must be assembled together.

On this background, the invention aims to offer a device which overcomes the aforementioned problems and offers the medical profession a device with few parts, cheap and perfectly suited to the practice and the requirements of f.i. vesical catheterization. The collection pouch according to the invention makes it possible in particular to collect and retain the liquid, even when a pressure or traction stress is applied to it.

To this end, the invention relates to a collection pouch for body fluids which is provided with an anti-reflux valve, said collection pouch being constituted of two sheets of plastic material, superposed and welded along their periphery, said collection pouch comprising:
a collection compartment;
an admission orifice; and
an admission channel located between said admission orifice and the entrance of said collection compartment, said admission channel being delineated by welds which constitute said anti-reflux valve, said collection pouch being characterised in that:
said admission orifice and said entrance to said collection compartment are offset, in particular axially, in relation to one another,
said welds of said admission channel define the internal limits of said collection compartment of the collection pouch,
said admission channel opens into said collection compartment distant from the lateral walls of the collection pouch.

In addition to the principal characteristics mentioned in the preceding paragraph, the device according to the invention may have one or more of the supplementary characteristics below, considered individually or in any of the technically possible combinations:
said admission channel extends into said collection compartment in such a way as to form, on at least one side of said entrance, a collection sub-compartment;
said admission channel is configured in such a way that a straight line joining the admission orifice and any point on the periphery of the collection compartment crosses at least one of said welds of said admission channel;
one of said welds is essentially parallel to the axis of said admission orifice;
said entry into said collection compartment is of a width between 14 and 20 mm;
said admission channel is of a width greater than or equal to 14 mm and in particular greater than or equal to 20 mm;
said welds, at the points of concentration of stresses, have a minimum width of 2 mm, in particular between 2 and 8 mm, preferably between 4 and 6 mm;
the borders of said welds are configured radially, i.e. they do not form any angles at which the pressure stresses might concentrate when the pouch is filled;
the width of said sub-compartment is greater than or equal to 20 mm, in particular between 20 and 40 mm, preferably between 25 and 35 mm, and the height of at least one sub-compartment is greater than or equal to 10 mm, in particular between 10 and 30 mm, preferably between 15 and 25 mm;
the lower extremities of said welds are aligned.

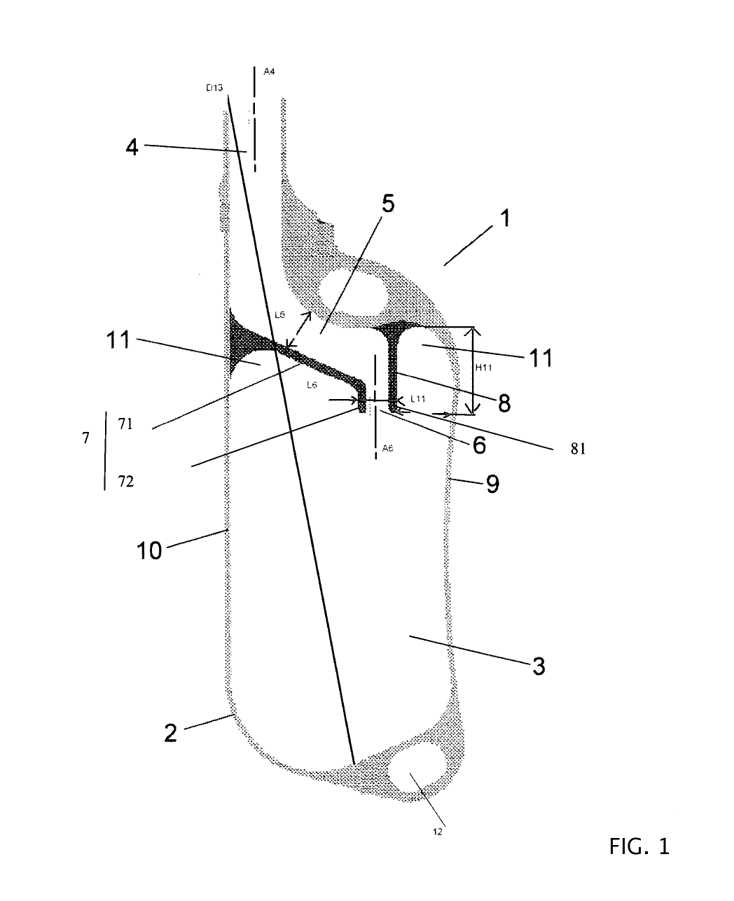

Other characteristics and advantages of the invention will emerge clearly from the description given below, which is by way of indication and in no way limiting, of the modes of realisation making reference to FIG. 1 attached, on which is represented an example of a device according to the invention.

For the sake of clarity, only the elements useful for the understanding of the invention have been shown, schematically and not to scale.

FIG. 1 illustrates a collection pouch 1 making it possible to collect any type of fluid, and in particular urine. This pouch comprises, notably:
an admission orifice 4;
an admission channel 5;
an entrance 6 to a collection compartment 3;
two welds 7 and 8;
two sub-compartments 11;
an eyelet 12;
two lateral welds 9 and 10 of the collection compartment 3, which form the lateral edges of the collection compartment 3.

Moreover, a straight line D13 is shown on FIG. 1.

The collection pouch 1 illustrated in FIG. 1 is formed of two sheets made, for example, of plastic material. During the assembly of the collection pouch 1, these sheets are superposed and welded along the whole of their periphery 2 such as to form a supple pouch. In other words, these two sheets by themselves form the bodily fluid collection pouch 1.

The welds 7 and 8 are realized between the two sheets of the collection pouch 1. These welds 7 and 8 may be, for example, heat-welds.

The weld 8 is essentially vertical (parallel to the axis A4) and comprises a lower extremity 81.

The weld 7 comprises a first oblique part 71 and a second essentially vertical part 72 (parallel to A4).

Advantageously, the lower extremities 72 and 81 of the welds 7 and 8 form the entrance 6 to the collection compartment 3 and finish at an essentially identical height.

Moreover, the two welds 7 and 8 form, on the one hand, an anti-reflux valve, the functioning of which is described below, and on the other hand, the lower part of the admission channel 5.

The anti-reflux valve, that is, the assembly formed by the welds 7 and 8, is offset axially from the admission orifice 4.

The first sub-compartment 11 is formed by the weld 10 and the weld 7.

The second sub-compartment 11 is formed by the weld 8 and the weld 9.

Note that the welds 9, 10 and the two welds 7 and 8 delimit the collection compartment 3.

Thus, when a liquid is introduced via the admission orifice 4, it runs down the admission channel 5 formed by the two welds 7, 8 and the two sheets. Consequently, the collection compartment 3 is filled via the intermediary of the admission channel 5.

In order to ensure uniform and regular flow, the width L5 of the admission channel 5 is preferably greater than or equal to 14 mm and in particular greater than or equal to 20 mm. Likewise, the width L6 of the anti-reflux valve is, preferably, between 14 and 20 mm.

When the fluids contained in the collection compartment 3 fill the sub-compartments 11, for example when the user of the pouch turns it upside-down, the anti-reflux valve closes and prevents any reflux of fluid through the admission channel 5. More specifically, when the sub-compartments 11 fill with fluid, the ensuing hydrostatic pressure is the same at all points of the walls and welds of these sub-compartments. The result of this hydrostatic pressure, however, is a specific configuration of tensions in the walls of the sub-compartments which tend to separate the welds 72 and 82 of the entrance 6 of said anti-reflux valve from one another and thereby to close this entrance 6, this closure becoming all the more hermetic as the pressure of fluid in the sub-compartments 11 increases. This closure prevents any reflux of liquid towards the exterior of the collection compartment 3, even if the collection pouch 1 is turned upside-down or subjected to any compression and/or traction stress.

In order to ensure that the anti-reflux valve of the collection pouch 1 is perfectly tight, the width L11 of the sub-compartments 11 measures between 20 and 40 mm, preferably between 25 and 35 mm, and the height H11 of said sub-compartments measures between 10 and 30 mm, preferably between 15 and 25 mm.

The volume formed by the width L11 and the height H11 corresponds to the minimum volume necessary to ensure the tightness of the anti-reflux valve.

Moreover, as shown in FIG. 1, the admission orifice 4, formed by the upper extremity of the admission channel 5, is offset axially from the entrance 6 to the collection compartment 3 formed by the lower extremity of the admission channel 5. This axial offsetting of the A4 and A6 axes makes it possible to maintain the tightness of the anti-reflux valve when the collection compartment 3 is filled and when a traction stress is exerted on the collection pouch 1 at the level of the eyelet 12 and of the admission orifice 4.

More specifically, this axial offset may, for example, be modelled by the straight line D13. This latter passes through the admission orifice 4 to join any point of the perimeter 2 of the two lateral welds 9 and 10 of the collection compartment 3, and must necessarily cut across one of the welds 7 or 8. This layout makes it possible to ensure that the anti-reflux valve remains tight, even if a traction or compression stress is applied to it.

Moreover, the weld 8 is essentially parallel to the axis A4 of the admission orifice 4. This particularity makes it possible to prevent the anti-reflux valve from opening when a traction effort is applied to the collection pouch 1.

In addition, the welds 7 and 8 are on the one hand configured radially, that is, they do not form any angles in which the pressure stresses might concentrate when the pouch is filled, and, on the other hand, have a width between 2 and 8 mm, preferably between 4 and 6 mm. This width is particularly important at the points of concentration of the stresses and avoids that the welds 7 and 8 are weakened and disrupt.

In short, the collection pouch 1 has in particular the advantage of offering a device comprising an anti-reflux valve that does not require any additional part or element, thus allowing the collection pouch 1 and the anti-reflux valve to be manufactured by a specific and ingenious combination of one or more welds of the two sheets of the body fluid collection pouch 1.

The collection pouch 1 according to the invention is tight when it is manipulated, and more specifically when a traction is applied to its two extremities.

The present invention has a direct application in the context of medical care, and in particular the field of managing urinary incontinence, whether in a hospital environment or in the community.

The invention is described in the above by way of example. It is understood that the person skilled in the art is able to carry out different variants of the collection pouch, in particular relating to the positioning or the layout of the welds that form the anti-reflux valve, without departing from the framework of the invention.

1. Collection pouch for body fluids provided with an anti-reflux valve, said collection pouch being constituted of two sheets of plastic material superimposed and welded along the whole of their periphery, said collection pouch comprising:
a collection compartment;
an admission orifice and;
an admission channel located between said admission orifice and an entrance to said collection compartment, said admission channel being defined by welds forming the anti-reflux valve, wherein
said admission orifice and said entrance to said collection compartment are offset in relation to one another,
said welds of said admission channel define the internal limits of said collection compartment of said collection pouch,
said admission channel opens into said collection compartment distant from the lateral walls of the collection pouch.

2. Collection pouch according to claim 1, wherein said admission channel extends into said collection compartment such as to form, on at least one side of said entrance, a collection sub-compartment.

3. Collection pouch according to the preceding claim 2, wherein the width of said sub-compartment is greater than or equal to 20 mm, and the height of said at least one sub-compartment is greater than or equal to 10 mm.

4. Collection pouch according to claim 1, wherein said admission channel is configured in such a way that a straight line joining said admission orifice and any point on the perimeter of said collection compartment crosses at least one of said welds of said admission channel.

5. Collection pouch according to claim 1, wherein one of said welds is essentially parallel to the axis of said admission orifice.

6. Collection pouch according to claim 1, wherein said entrance to said collection compartment has a width between 14 and 20 mm.

7. Collection pouch according to claim 1, wherein said admission channel has a width greater than or equal to 14 mm.

8. Collection pouch according to claim 1, wherein the lower extremities of said welds are aligned.

9. Collection pouch according to claim 1, wherein said welds have, at the points of concentration of stresses, a minimum width of 2 mm.

10. Collection pouch according to claim 1, wherein said welds are configured radially.

11. Collection pouch according to claim 2 wherein the width of said sub-compartment is between 20 and 40 mm.

12. Collection pouch according to claim 2 wherein the width of said sub-compartment is between 25 and 35 mm.

13. Collection pouch according to claim 2 wherein the height of said at least one sub-compartment is between 10 mm and 30 mm.

14. Collection pouch according to claim 2 wherein the height of said at least one sub-compartment is between 15 mm and 25 mm.

15. Collection pouch according to claim 1, wherein said admission channel has a width greater than or equal to 20 mm.

16. Collection pouch according to claim 1, wherein said welds have, at the points of concentration of stresses, a minimum width of between 2 and 8 mm.

17. Collection pouch according to claim 1, wherein said welds have, at the points of concentration of stresses, a minimum width of between 4 and 6 mm.
